(19) United States
(12) Patent Application Publication
(10) Pub. No.: US 2005/0220861 A1
(43) Pub. Date: Oct. 6, 2005

(54) Colonic release compostion

(76) Inventors: Richard Michael John Palmer, CAMBRIDGE (GB); Mike Newton, London (GB); Abdul Basit, Middlesex (GB); John Bloor, Cambridgeshire (GB)

Correspondence Address: CHOATE, HALL & STEWART LLP, TWO INTERNATIONAL PLACE, BOSTON, MA, 02110, US

(21) Appl. No.: 10/504,182

(22) PCT Filed: Feb. 13, 2003
(86) PCT No.: PCT/GB03/00651

§ 371 Date: Apr. 25, 2005

(30) Foreign Application Priority Data
Feb. 13, 2002 (GB) 0203421.3

(51) Int. Cl. A61K 9/48; A61K 9/26

(52) U.S. Cl. 424/451; 424/469

(57) ABSTRACT

The present invention relates to an improved controlled (preferably delayed) release formulation for delivery of prednisolone sodium metasulphobenzoate. The formulation comprises prednisolone sodium metasulphobenzoate surrounded by a coating comprising glassy amylose, ethyl cellulose and dibutyl sebacate, wherein the ratio of amylose to ethyl cellulose is from (13.5) to (1:4.5) and wherein the amylose is corn or maize amylose.

The present invention relates to an improved controlled (preferably delayed) release formulation for delivery of prednisolone sodium metasulphobenzoate. The formulation comprises prednisolone sodium metasuiphobenzoate surrounded by a coating comprising glassy amylose, ethyl cellulose and dibutyl sebacate, wherein the ratio of amylose to ethyl cellulose is from 1:3.5 to 1:4.5 and wherein the amylose is corn or maize amylose.

The colon can be a site for the local action and/or, potentially, the systemic absorption of therapeutic agents. Another advantage is that actives can be selectively taken up in the colon. The delivery of drugs directly to their site of action in the treatment of diseases such as inflammatory bowel diseases (IBD), can increase their efficacy and reduce unpleasant and/or serious side effects that result from systemic absorption.

There are a number of systems that attempt to deliver drugs to the colon. In general, these are limited in their performance and/or specificity and can be characterised as
rectally-delivered foams and enemas
pH-triggered, oral delivered systems
pro-drugs which are activated in the colon
time-dependent oral delivered systems.

The site of activity of rectally administered products is generally limited to the rectum and distal colon, and patient acceptability is a problem.

Orally delivered products that rely on pH and/or time dependent-mechanisms for drug release are inherently unreliable in attaining consistent colon-specific delivery in patients. This is due to the wide variability in transit times and pH differentials in the various parts of the gut.

The recent development of oral delivery systems that use bacterial enzymes to trigger the release of actives offers the potential to overcome many of the problems experienced with the earlier systems.

In WO 91/07949, a delayed release formulation is described for general application in targeting medicaments and diagnostic agents into the colon.

However, this document does not take into account, or teach, that different actives can have individual formulations to obtain the optimum targeting to the desired site and absorption profiles. Since medicinal actives differ in a number of biochemical and biophysical characteristics, such as absorption, polarity, solubility and logp, it may be possible to provide individual formulations which provide such optimisation.

The present invention provides such a formulation for the active prednisolone sodium metasulphobenzoate.

Prednisolone sodium metasulphobenzoate is a corticosteroid, known for its use in treating Inflammatory Bowel Disease (IDB). It is generally poorly absorbed from the upper gastrointestinal tract and is currently administered rectally as a topical formulation. This is often unpleasant and unpopular with patients. It is also difficult to control dose delivery and only the distal part of the colon can be reached.

The present invention provides a novel formulation for improved controlled release of an oral dose of prednisolone sodium metasulphobenzoate.

A first aspect of the invention provides a controlled release formulation comprising prednisolone sodium metasulphobenzoate surrounded by a coating comprising glassy amylose, ethyl cellulose and dibutyl sebacate. The amylose and the ethyl cellulose are plasticised with the dibutyl sebacate. The ratio of amylose to ethyl cellulose is from 1:3.5 to 1:4.5. The amylose is corn or maize derived. The controlled release formulation is preferably delayed release.

This formulation provides an advantageous delivery of the prednisolone sodium metasulphobenzoate to the colon. The delivery of prednisolone sodium metasulphobenzoate is coincident with the arrival of the dosage form in the colon.

The characteristics of glassy amylose are well known and are described, e.g. in WO 91/07949. As described in WO 91/07949 and as applicable to the present invention, the glassy amylose preferably has a Tg of not less than 20° C. below the temperature at which the use of the composition is carried out. This temperature (at which the composition is used) will usually be body temperature, i.e. 37° C. The Tg is thus preferably around 17° C. or more. It may be around 25° C. or more, around 30° C. or more, or 35° C. or more. Controlling the amount of water in the amylose composition predetermines the Tg. This can be carried out by a number of procedures known in the art, such as the concentration of amylose in the solution as well as spraying and drying of the resulting product.

The amylose may be prepared by any technique known in the art, such as by forming a gel from an aqueous solution and then drying or by spray drying.

The resulting dry glassy amylose can be further processed. It may be melted (in the form of a slab) or may first be powdered or granulated. After such melting, the amylose can be used to coat pellets, or other forms, of active ingredient.

Typically, the amylose is 1 to 15%, preferably 2 to 10%, or 3 to 5% of the solution (on a weight by weight basis). As described below, the solution may be aqueous or an aqueous-alcohol mix.

The glassy amylose is part of the coating, in combination with ethyl cellulose and dibutyl sebacate. Typically, a solution of around 15 to 20% ethyl cellulose is admixed with the other ingredients. The final range of ethyl cellulose in the coating product is usually in the range of from 2 to 15%, preferably from 5 to 10% on a weight by weight basis.

The amylose, ethyl cellulose and dibutyl sebacate are preferably admixed before applying to the prednisolone sodium metasulphobenzoate.

It is preferable that the glassy amylose comprises as little moisture as possible. It should be lower than 20% (w/w), more preferably lower than 15% (w/w).

It has been determined that the particular combination of glassy amylose, ethyl cellulose and dibutyl sebacate provides the optimum colonic delivery formulation for prednisolone sodium metasulphobenzoate. It is thus preferred that the presence of any other ingredient in the coating is minimised to no more than 10% (w/w). Furthermore, any hydroxy group in derivative form, of the amylose, should be limited to no more than 10% of the hydroxy groups present. A convenient test for the purity of amylose can be found in Banks et al, Starke, 1971, 23,118.

The preferred ratios of the three components are, on a weight by weight basis of:

glassy amylose:ethyl cellulose:dibutyl sebacate

1:3.5 to 4.5:0.5 to 1.5
1:3.5 to 4.5:0.8 to 0.9
1:3.5 to 4.5:0.85
1:4:0.85

Suitable dosage forms of the present invention include-prednisolone sodium metasulphobenzoate (expressed as prednisolone) at 20 mg, 40 mg, 60 mg, 80 mg, 100 mg or 120 mg (per day). An example of a suitable range of from 40 mg to 120 mg per day can be used for treatment. An example of a suitable range of from 40 to 60, 70, 80 or 100 mg per day can be used for prevention and/or treatment of inflammatory bowel disease. By prevention, we particularly include maintenance of remission.

The prednisolone sodium metasulphobenzoate is usually admixed with a filler. The filler may be any suitable agent, for example comprising or consisting of one or more of lactose, mannitol, sorbitol, xylitol, starch, or a cellulose derivative. In the present invention, the filler preferably is or comprises mannitol or lactose. The mannitol preferably has a mean particle size of around 85-90 μm and a bulk density of around 0.66 gcm⁻³. The lactose preferably has a mean particle size such that 95% of particles are less than 45 μm. Preferably the lactose has a bulk density of around 0.47 gcm⁻³. The mannitol or lactose preferably present in the range of ratios from 1:5 to 1:2 with the prednisolone sodium metasulphobenzoate. This “core” comprising the active ingredient, may also comprise microcrystalline cellulose, in order to optimise extrusion and spheronisation. The ratio of microcrystalline cellulose to prednisolone sodium metasulphobenzoate is in the range of 1:2.5 to 1:0.5, preferably 1:2.5 to 1:1.1, preferably approximately 1:1.2.

The formulation of the present invention is most preferably in the form of pellets, tablets, mini-tabs or capsules. In each formulation, the coating thickness equates to around 15% to 25% of the total weight of the formulation. The pellet formulation may range in size, for example from 0.5 to 1.5 mm in diameter.

In the present invention, dibutyl sebacate has been determined to provide an optimum combination of plasticiser function and drug release. In the selection of plasticiser, dibutyl sebacate was found to be preferable as follows:—

A plasticiser of fractionated coconut oil resulted in diffusion/digestion problems. A plasticiser of dibutyl sebacate containing silica provided too high a diffusion element of the diffusion/digestion release profile. Use of dibutyl sebacate was used to provide optimum functionality of digestion and minimised drug diffusion prior to digestion of the coat.

The formulation according to the invention may be within a capsule. Such a capsule may be any known in the art, such as a capsule comprising one or more of gelatin, starch or hydroxypropylmethyl cellulose.

The second aspect of the invention provides a process for producing a formulation according to the first aspect of the invention. Any process known in the art can be used. As described above, glassy amylose must first be prepared. This anylose can then be applied to the active “core” in layers or otherwise. Preferably, the amylose is admixed with the ethyl cellulose and dibutyl sebacate before applying to the active “core”. Dry glassy amylose can be melted in the form of a slab or film or can first be granulated or powdered. The melted amylose is then mixed with the ethyl cellulose and dibutyl sebacate before being applied to the active “core”. Alternatively, an aqueous alcoholic or an aqueous solution of amylose, is optionally admixed with ethyl cellulose and dibutyl sebacate and can be applied to the active “core”. In this process, the concentration of amylose in the solution is usually in the region of 1 to 15%, or preferably 1 to 10%, or most preferably 1 to 5% (weight by weight). Typically, the coating is applied to the active by spraying or dipping. Suitable spraying and dipping machines are well known in the art and can be used in the process of the present invention.

In particular, ethyl cellulose in aqueous media is applied directly to a 20% amylose suspension in aqueous ammonia. Mixtures of ethyl cellulose and amylose are preferably prepared by mixing in the ratio of 4:1 with the temperature maintained above 60° C. during the coating process. The resulting product is dried for one hour at 60° C.

A process as described in WO 99/21536 can also be used in the present invention. This method provides contacting the active “core” with a solution of the coating composition in a solvent system comprising both water and a water miscible organic solvent. The water miscible organic solvent being capable, on its own, of dissolving ethyl cellulose. The water and organic solvent are then removed. The solvent system should contain at least 50% weight by weight organic solvent. Contrary to the process described in WO 99/21536, the proportion of amylose to film-forming polymer can be any of those described in the present invention. In this process, the temperature can be any ranging from 20° C. upwards, in particular in the range of 20° C. to 50° C. or 60° C., although a temperature of over 60° C. can also be used. Again, application of the coating to the active material is preferably by spraying or by dipping, although the process is not limiting.

Furthermore, a process as described in WO 99/25325 can be used according to the present invention. This method provides a method of coating active material with a coating comprising an aqueous dispersion of an amylose alcohol mix, ethyl cellulose and a plasticiser at a temperature of less than 60° C. The coating, preferably contains between 1 and 15% weight by weight of amylose alcohol mix. The coating compositions are prepared by admixing an aqueous dispersion of an amylose alcohol mix with an aqueous dispersion of the ethyl cellulose and dibutyl sebacate. Usually, the aqueous dispersion of the ethyl cellulose is pre-plasticised by rapid, sheer mixing with an aqueous dispersion of the plasticiser. Alternatively, the ethyl cellulose and the plasticiser can be directly mixed. The aqueous dispersion of the amylose alcohol mix is preferably a dispersion of an amylose butanol mix. Usually, the concentration of the amylose butanol mix in a dispersion is in the range of 1 to 15% weight by weight of the final dispersion.

Following application of the coating to the active “core”, the composition is dried. The formulation can be allowed to dry in air or in an inert atmosphere. Alternatively, the formulation can be dried by curing. The curing may be carried out at a temperature of between 5° C. and 60° C. over a period of up to 6 hours, preferably around 1 hour at approximately 60° C. Longer curing times are preferably avoided as these may result in crystalline regions within the coating. Shorter curing times ensure that the amylose is retained in the glassy form. After curing, the final products are preferably packaged such that they are protected from moisture.

All preferred features of the first aspect of the invention, also apply to the second aspect.

A third aspect of the present invention provides a formulation according to the first aspect of the invention, for use in the prevention or treatment of inflammatory bowel disease. In the present invention, inflammatory bowel disease includes Crohn's colitis and ulcerative colitis.

In the present invention, “prevention” includes maintaining a patient in a disease free state or maintaining a patient with low-level (eg tolerable) symptoms.

All preferred features of the first and second aspects also apply to the third aspect.

In a fourth aspect of the invention, there is provided the use of glassy amylose, ethyl cellulose, dibutyl sebacate and prednisolone sodium metasulphobenzoate, in the manufacture of a medicament for the prevention or treatment of inflammatory bowel disease.

All preferred features of the first to third aspects of the invention also apply to the fourth aspect.

The present invention refers to the following Figures:

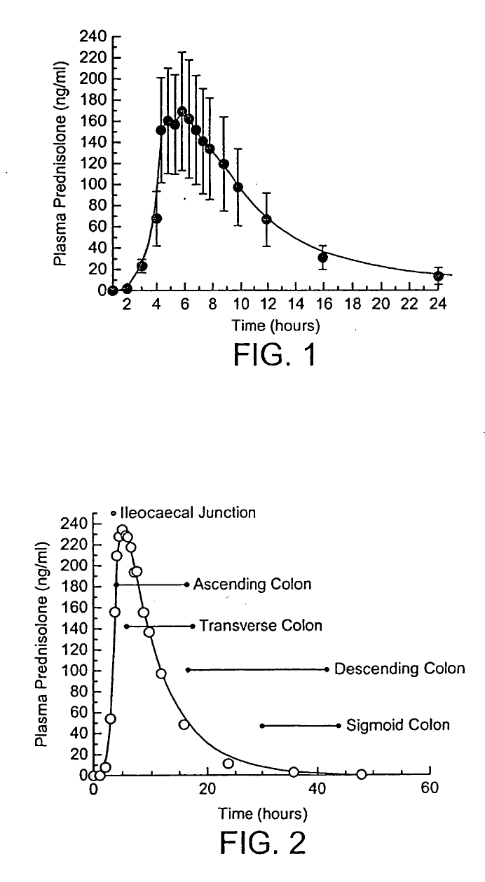

FIG. 1: Plasma prednisolone drug levels after administration of coated prednisolone sodium metasulphobenzoate (equivalent to 60 mg prednisolone).

Each point is the mean±standard error of the mean for seven subjects.

FIG. 2: Plasma prednisolone drug levels in Subject 2 after administration of coated prednisolone sodium metasulphobenzoate (equivalent to 60 mg prednisolone), showing drug release when the pellets are localised in the colon.

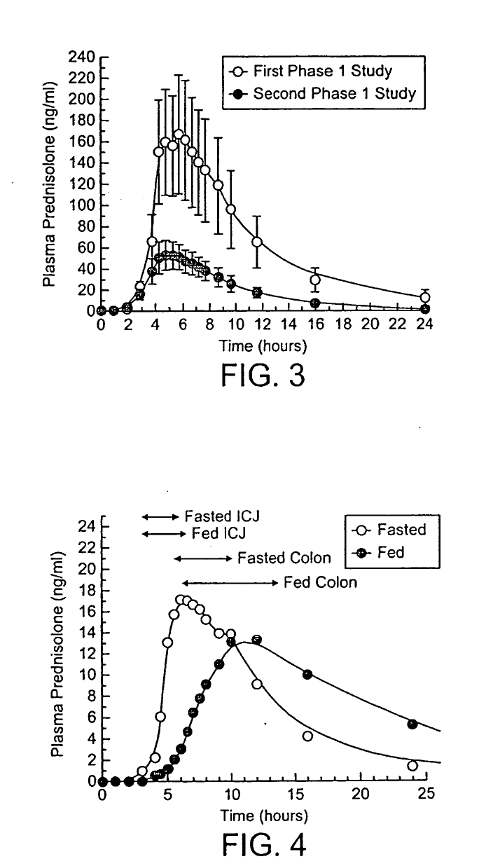

FIG. 3: Plasma prednisolone drug levels after administration of prednisolone sodium metasulphobenzoate (equivalent to 60 mg prednisolone), in different coatings.

FIG. 4: Plasma prednisolone drug levels after administration of prednisolone sodium metasulphobenzoate (equivalent to 60 mg prednisolone), in fed and fasted subjects.

The present invention is now described with reference to the following, non-limiting examples.

### EXAMPLES

### Example 1

Pellet Production—Lactose Filler

Pellets of prednisolone sodium metasulphobenzoate, microcrystalline cellulose and lactose (47% prednisolone sodium metasulphobenzoate, 40% microcrystalline cellulose, 13% lactose) were reliably and efficiently produced by a process of extrusion through a die or mesh, followed by spheronisation, achieved through breakage and rounding on a hatched plate, rotating in a cylinder. Successful extrusion-spheronisation required the production of a cohesive wet mass which flowed through the die without adhering to the extruder or to itself, whilst retaining a degree of rigidity so that the shape imposed by the die is retained. Furthermore the extrudate must be brittle enough to break into uniform lengths on the spheronisation plate, yet still be plastic enough to round into spherical pellets.

The pellets produced had an acceptable appearance, strength, friability and release characteristics.

Delayed Release Formulation Production

A mixed polymer suspension containing maize amylose, ethyl cellulose and dibutyl sebacate (in the ratio of 1:4:0.85) was heated to convert the amylose into its amorphous form. The resulting solution was sprayed into the top of a fluidised bed of the pellets prepared above (lactose filler), until a 20% total weight gain was obtained.

The coated product was then cured for approximately 1 hour at 60° C., in air. The coated pellets were filled into a hard gelatin capsule.

### Example 2

Pellet Production—Mannitol Filler

Pellets of prednisolone sodium metasulphobenzoate, microcrystalline cellulose and mannitol (47% prednisolone sodium metasulphobenzoate, 40% microcrystalline cellulose, 13% mannitol) were reliably and efficiently produced by a process of extrusion through a die or mesh, followed by spheronisation, achieved through breakage and rounding on a hatched plate, rotating in a cylinder. Successful extrusion-spheronisation required the production of a cohesive wet mass which flowed through the die without adhering to the extruder or to itself, whilst retaining a degree of rigidity so that the shape imposed by the die is retained. Furthermore the extrudate must be brittle enough to break into uniform lengths on the spheronisation plate, yet still be plastic enough to round into spherical pellets.

The pellets produced had an acceptable appearance, strength, friability and release characteristics consistent with colon targetting.

Delayed Release Formulation Production

A mixed polymer suspension containing maize amylose, ethyl cellulose and dibutyl sebacate (in the ratio of 1:4:0.85) was heated to convert the amylose into its amorphous form. The resulting solution was sprayed into the top of a fluidised bed of the pellets prepared above (mannitol filler), until a 20% total weight gain was obtained.

The coated product was then cured for approximately 1 hour at 60° C., in air. The coated pellets were filled into a hard gelatin capsule.

### Example 3

Phase I Study of Colon Targeting

Using pellets described in Example 1.

Four phase I studies have been completed. In the first of these, an ethyl cellulose to amylose ratio of 5:1 was used with a 10% weight gain. The second and third studies, investigated the effect of a thicker coat (20% weight gain) at a 4:1 ethylcellulose to amylose ratio. All other features of the comparison formulation were the same.

Capsules containing coated pellets of prednisolone sodium metasulphobenzoate, equivalent to 60 mg prednisolone, were administered to seven healthy, fasted volunteers. Progress of the dose through the gastrointestinal tract was followed over 24 hours by gamma-scintigraphy after the co-administration of ethylcellulose coated ¹¹¹indium labelled non-pareils. Plasma levels of prednisolone were determined at various time points up to 48 hours after administration. Excreted drug pellets were harvested over five days and residual drug levels determined.

Plasma prednisolone levels appeared at around two hours after dosing, rising to a maximum at between five and six hours. The mean Cₘₐₓ was lower than those reported in two of three patients following treatment with a 60 mg prednisolone metasulphobenzoate enema and the data were generally rather less variable than those reported by McIntyre et al (1985). Although the mean AUCs were higher than reported by McIntyre et al, they were substantially lower than those reported for oral administration of lower doses.

Comparison of prednisolone Cₘₐₓ and AUC from the formulation with conventional
oral and rectal formulations of the metasulphobenzoate and with the 21-phosphate
given rectally.

| Study | Compound | Dose, route | Cₘₐₓ | AUC |
|---|---|---|---|---|
| Study 1 | Prednisolone | 60 mg, oral | 168 ± 56 | 1441 ± 541 |
| Amylose:Ethlycellulose | metasulphobenzoate | | | |
| 1:5 | | | | |
| 10% Weight Gain | | | | |
| McIntyre et al | Prednisolone | 60 mg, rectal | 147 ± 79 | 632 ± 509 |
| 1985 | metasulphobenzoate | | | |
| | Prednisolone-21-phosphate | 20 mg, rectal | 148 ± 75 | 599 ± 310 |
| Flouvat et al 1991 | Prednisolone | 40 mg, oral | 242 ± 58 | 2189 ± 475 |
| | metasulphobenzoate | | | |

The data indicate that this formulation is targeted to the proximal colon, although the plasma concentrations are substantially lower than those reported for conventional oral dose forms.

The plasma-time curve is plotted in FIG. 1.

The spread of the pellets after disintegration of the capsule was considerable and varied between subjects. However, a substantial proportion of the bioavailability occurred when the majority of the coated pellets had reached the ileo-caecal junction and proximal colon, illustrated in FIG. 2 for one of the subjects.

The amount of prednisolone recovered from pellets harvested from the faeces was low and similar in all subjects, averaging 2.5±1.12 mg and being less than 5% of the administered dose in all cases.

### Example 4

Second Phase I Study of Colon Targeting—Example According to the Invention

The trial comprised a combined gamma scintigraphic and pharmacokinetic study with excreted pellet analysis to the same design as the first Phase I study (above) following administration of the prednisolone sodium metasulphobenzoate ester at a dose of 94.2 mg, equivalent to 60 mg of prednisolone. In this second study, the thickness of the coating of the pellets was increased and the proportion of amylose was increased in the coat. This reduced drug release and consequent absorption in the small intestine, and gave improved colon targeting through an enhanced opportunity for digestion by colonic microbial amylases.

Comparison of prednisolone Cₘₐₓ and AUC from two formulations with a
conventional enema.

| | | | Cₘₐₓ | AUC |
|---|---|---|---|---|
| Study | Compound | Dose, route | ng/ml | ng · ml · h |
| 1st Phase 1 | Prednisolone | 60 mg, oral | 168 ± 56 | 1441 ± 541 |
| Amylose:Ethlycellulose | metasulphobenzoate | | | |
| 1:5, Weight | | | | |
| Gain 10% | | | | |
| 2nd Phase 1 | Prednisolone | 60 mg, oral | 54 ± 15 | 395 ± 105 |
| Amylose:Ethylcellulose | metasulphobenzoate | | | |
| 1:4 Weight | | | | |
| Gain 20% | | | | |
| McIntyre et al | Prednisolone | 60 mg, rectal | 147 ± 79 | 632 ± 509 |
| 1985 | metasulphobenzoate | | | |

The peak prednisolone plasma levels, determined over 24 hours, were significantly lower than those determined in the previous study. This reduction in plasma level is a consequence of more specific colonic targeting with lower release in the ileum, due to the coat reformulation and increase in coat weight on the pellets.

Comparisons of plasma level data and gamma scintigraphy images in FIG. 3 show that the majority of the limited drug absorption occurred at or after the ileo-caecal junction, confirming that the coating of the present invention provides an optimal effective and specific colonic delivery system.

As in the first Phase I study, the time to peak plasma levels of drug coincided almost exactly with arrival of the drug pellets at the ileo-caecal junction (5.9±0.4 hours vs 5.9±2.0 hours) with rather more variation in arrival time, again reflecting the variable transit time of pellets within the gastro-intestinal tract. This was again indicative of colonic release, regardless of transit time, through amylose digestion.

Despite the low systemic bioavailability in the second Phase I study, the quantity of prednisolone harvested from excreted pellets in the faeces was low in all subjects as with the first Phase I study. The mean quantity of drug excreted in the pellets was 1.7 mg+0.37, determined as prednisolone sodium metasulphobenzoate, this being less than 2% of the administered dose. This suggests that the vast majority of prednisolone metasulphobenzoate had been released, and was available, for local action in the colon.

### Example 5

Third Phase I Study

The trial comprised a combined gamma scintigraphic and pharmacokinetic study with excreted pellet analysis to the same design as the first Phase I study (above) following administration of prednisolone sodium metasulphobenzoate ester at a dose of 94.2 mg, equivalent to 60 mg prednisolone. In this third study, administering the coated prednisolone pellets to volunteers in the fed and fasted state was examined. The presence of food increased gastrointestinal transit time.

Comparison of prednisolone Cₘₐₓ and AUC from the Third Phase 1 Study
(Amylose: Ethylcellulose 1:4, 20% Coat Weight Gain)

| | | | Cₘₐₓ | AUC |
|---|---|---|---|---|
| Study | Compound | Dose, route | ng/ml | ng · ml · h |
| 3^{rd} phase I | Prednisolone | 60 mg, oral | 17 | 213 |
| Fed | metasulphobenzoate | | | |
| 3^{rd} phase I | Prednisolone | 60 mg, oral | 14 | 185 |
| Fasted | metasulphobenzoate | | | |

Administration of the dosage form with food had no effect on the initial disintegration of the capsule in the stomach. Gastrointestinal transit times were extended in the fed state compared to the fasted state (Table below).

Gastrointestinal Transit for Radiolabelled Pellets

| | Time (hours) | | | | | |
|---|---|---|---|---|---|---|
| Pellet Transit | Fasted | | | Fed | | |
| Profile | Mean | SD | Median | Mean | SD | Median |
| ICJ Arrival | 4.67 | 2.10 | 4.36 | 4.52 | 3.42 | 2.82 |
| ICJ Complete | 6.90 | 1.90 | 6.63 | 7.86 | 2.25 | 7.00 |
| Colon Arrival | 5.57 | 2.31 | 4.65 | 6.34 | 2.81 | 6.02 |
| Colon | 10.72 | 4.49 | 9.40 | 13.85 | 6.01 | 11.69 |
| Complete | | | | | | |

Release of drug, as indicated by a rise in plasma levels, occurred when the pellets reached the ileocaecal junction (ICJ) or ascending colon (FIG. 4), regardless of the time taken to reach this location and the feeding state.

Despite the low plasma levels observed in this study, excreted pellet analysis showed that, in both the fed and fasted study, over 90% of the drug content of the formulation had been released from the pellets and was available for local action in the colon. The mean value for the prednisolone sodium metasulphobenzoate retained in the excreted pellets from the fasted study was 6.1±1.0 mg and in the fed study the value was 3.1±1.2 mg.

### Example 6

Fourth Phase I Study

The pellets were produced as described in Example 2.

This trial used a higher dose of prednisolone sodium metasulphobenzoate, equivalent to 100 mg of prednisolone. The increase in dose produced little effect on the peak plasma level or the area under the plasma curve, when compared to the previously administered 60 mg dose. This study indicates that higher doses may be used to deliver prednisolone to the colon, with low systemic absorption and consequent low risk of systemic adverse events.

Comparison of prednisolone Cₘₐₓ and AUC from the Fourth Phase 1 Study

| | | | Cₘₐₓ | AUC |
|---|---|---|---|---|
| Study | Compound | Dose, route | ng/ml | ng · ml · h |
| 4^{th} phase I | Prednisolone | 100 mg, | 18.9 | 152 |
| Fasted | metasulphobenzoate | oral | | |

Discussion

The present invention is a new formulation which provides good targeted release of prednisolone sodium metasulphobenzoate to the colon, with low systemic exposure. This formulation thus allows the treatment and prevention of Inflammatory Bowel Diseases, such as ulcerative colitis and Crohn's disease with prednisolone sodium metasulphobenzoate, without systemic side effects The present formulation provides an improved colonic delivery system over the known art.

1. A controlled release formulation comprising prednisolone sodium metasulphobenzoate surrounded by a coating comprising glassy amylose, ethyl cellulose and dibutyl sebacate, wherein the ratio of amylose to ethyl cellulose is from 1:3.5 to 1:4.5 and wherein the amylose is corn or maize amylose.

2. A formulation, as claimed in claim 1, wherein the prednisolone sodium metasulphobenzoate is admixed with a filler.

3. A formulation, as claimed in claim 2, wherein the filler is mannitol or lactose.

4. A formulation, as claimed in any one of claims 1 to 3, wherein the coating thickness is 15 to 25% of the total weight of the formulation.

5. A formulation, as claimed in any one of claims 1 to 3, which is the form of a pellet, tablet, mini-tab or capsule.

6. A formulation, as claimed in any one of claims 1 to 3, which is from 0.5 to 1.5 mm in diameter.

7. A formulation, as claimed in any one of claims 1 to 3, wherein the ratio of amylose, ethyl cellulose and dibutyl sebacate is in the range of 1:3.5 to 4.5:0.5 to 1.5.

8. A process for producing a formulation, the process comprising admixing glassy amylose, ethyl cellulose and dibutyl sebacate and applying the admixture as a coating to a core of prednisolone sodium metasulphobenzoate, wherein the ratio of amylose to ethyl cellulose in the formulation is from 1:3.5 to 1:4.5 and wherein the amylose is corn or maize amylose.

9. A method for preventing or treating Inflammatory Bowel Disease, the method comprising administering to a patient a formulation as claimed in claim 1.

10. Use of glassy amylose, ethyl cellulose, dibutyl sebacate and prednisolone sodium metasulphobenzoate, in the manufacture of a medicament for the prevention or treatment of Inflammatory Bowel Disease.

11. A formulation, as claimed in any one of claims 1 to 3, wherein the formulation is within a capsule.

12. A formulation, as claimed in claim 11, wherein the capsule comprises one or more of gelatin, starch or hydroxypropylmethyl cellulose.

13. A formulation, as claimed in claim 4, wherein the ratio of amylose, ethyl cellulose and dibutyl sebacate is in the range of 1:3.5 to 4.5:0.5 to 1.5.

14. A formulation, as claimed in claim 5, wherein the ratio of amylose, ethyl cellulose and dibutyl sebacate is in the range of 1:3.5 to 4.5:0.5 to 1.5.

15. A formulation, as claimed in claim 6, wherein the ratio of amylose, ethyl cellulose and dibutyl sebacate is in the range of 1:3.5 to 4.5:0.5 to 1.5.

16. A process, as claimed in claim 8, wherein the prednisolone sodium metasulphobenzoate is admixed with a filler.

17. A process, as claimed in claim 16, wherein the filler is mannitol or lactose.

18. A process, as claimed in any one of claims 8, 16 or 17, wherein the coating thickness is 15 to 25% of the total weight of the formulation.

19. A process, as claimed in any one of claims 8, 16 or 17, wherein the formulation is the form of a pellet, tablet, mini-tab or capsule.

20. A process, as claimed in any one of claims 8, 16 or 17, wherein the formulation is from 0.5 to 1.5 mm in diameter.

21. A process, as claimed in any one of claims 8, 16 or 17, wherein the ratio of amylose, ethyl cellulose and dibutyl sebacate in the formulation is in the range of 1:3.5 to 4.5:0.5 to 1.5.

22. A process, as claimed in claim 18, wherein the ratio of amylose, ethyl cellulose and dibutyl sebacate in the formulation is in the range of 1:3.5 to 4.5:0.5 to 1.5.

23. A process, as claimed in claim 19, wherein the ratio of amylose, ethyl cellulose and dibutyl sebacate in the formulation is in the range of 1:3.5 to 4.5:0.5 to 1.5.

24. A process, as claimed in claim 20, wherein the ratio of amylose, ethyl cellulose and dibutyl sebacate in the formulation is in the range of 1:3.5 to 4.5:0.5 to 1.5.

25. A method, as claimed in claim 9, wherein the prednisolone sodium metasulphobenzoate is admixed with a filler.

26. A method, as claimed in claim 25, wherein the filler is mannitol or lactose.

27. A method, as claimed in any one of claims 9, 25 or 26, wherein the coating thickness is 15 to 25% of the total weight of the formulation.

28. A method, as claimed in any one of claims 9, 25 or 26, wherein the formulation is the form of a pellet, tablet, mini-tab or capsule.

29. A method, as claimed in any one of claims 9, 25 or 26, wherein the formulation is from 0.5 to 1.5 mm in diameter.

30. A method, as claimed in any one of claims 9, 25 or 26, wherein the ratio of amylose, ethyl cellulose and dibutyl sebacate in the formulation is in the range of 1:3.5 to 4.5:0.5 to 1.5.

31. A method, as claimed in claim 27, wherein the ratio of amylose, ethyl cellulose and dibutyl sebacate in the formulation is in the range of 1:3.5 to 4.5:0.5 to 1.5.

32. A method, as claimed in claim 28, wherein the ratio of amylose, ethyl cellulose and dibutyl sebacate in the formulation is in the range of 1:3.5 to 4.5:0.5 to 1.5.

33. A method, as claimed in claim 29, wherein the ratio of amylose, ethyl cellulose and dibutyl sebacate in the formulation is in the range of 1:3.5 to 4.5:0.5 to 1.5.
